(19) United States
(12) Patent Application Publication
(10) Pub. No.: US 2017/0119322 A1
(43) Pub. Date: May 4, 2017

(54) MEDICAL IMAGE DIAGNOSIS SYSTEM, STRUCTURAL IMAGE DIAGNOSIS APPARATUS, AND NUCLEAR MEDICAL IMAGE DIAGNOSIS APPARATUS

(71) Applicant: Toshiba Medical Systems Corporation, Otawara-shi (JP)

(72) Inventors: Yasunobu YAMADA, Nasushiobara (JP); Yasuhiro NOSHI, Otawara (JP)

(73) Assignee: Toshiba Medical Systems Corporation, Otawara-shi (JP)

(21) Appl. No.: 15/341,353

(22) Filed: Nov. 2, 2016

(30) Foreign Application Priority Data
Nov. 2, 2015 (JP) 2015-216150
Oct. 31, 2016 (JP) 2016-213666

(51) Int. Cl. A61B 6/04; A61B 6/00; A61B 6/03

(52) U.S. Cl. CPC A61B 6/0457; A61B 6/035; A61B 6/037; A61B 6/0407; A61B 6/4007; A61B 6/547; A61B 6/5205

(57) ABSTRACT

A medical image diagnosis system according to an embodiment includes a structural image diagnosis apparatus and a nuclear medical image diagnosis apparatus. The system includes a processing circuit that acquires information on a position of a body part of a subject based on a structural image acquired by the structural image diagnosis apparatus and a gantry that scans a nuclear medical image of the subject. The processing circuit controls the gantry based on the information on the position of the body part and a scan condition of the body part.

### CROSS-REFERENCE TO RELATED APPLICATIONS

This application is based upon and claims the benefit of priority from Japanese Patent Application No. 2015-216150, filed on Nov. 2, 2015; and Japanese Patent Application No. 2016-213666, filed on Oct. 31, 2016, the entire contents of all of which are incorporated herein by reference.

### FIELD

Embodiments described herein relate generally to a medical image diagnosis system, a structural image diagnosis apparatus, and a nuclear medical image diagnosis apparatus.

### BACKGROUND

Recently, there is known an apparatus, such as a Positron Emission Tomography-Computed Tomography apparatus (PET-CT apparatus), in which a functional image diagnosing apparatus and a structural image diagnosis apparatus are combined.

In a medical image diagnosis system in which a structural image diagnosis apparatus and a functional image diagnosis apparatus are combined, for example, the structural image diagnosis apparatus performs a scan to generate a localizer image, and the functional image diagnosis apparatus performs a whole-body scan, by a scan plan planned by an operator, on the basis of the generated localizer image.

However, in such a case that the functional image diagnosis apparatus concurrently performs a whole-body scan and a detailed scan of a specific body part, or detailed scans of a plurality of body parts, the operator needs to input scan time duration of the functional image diagnosis apparatus for each scan position while checking on the scanned localizer image the scanning range and the body part. Therefore, there remains a heavy burden on the operator.

### BRIEF DESCRIPTION OF THE DRAWINGS

FIG. 1 is a diagram illustrating an overall configuration of a medical image diagnosis system according to a first embodiment;

FIG. 2 is a block diagram illustrating the overall configuration of the medical image diagnosis system according to the first embodiment;

FIG. 3 is a flowchart illustrating a processing procedure of the medical image diagnosis system according to the first embodiment;

FIG. 4 is a diagram illustrating setting of a scan condition of the medical image diagnosis system according to the first embodiment;

FIG. 5 and FIGS. 6A to 6C are diagrams illustrating setting of a scan condition of a medical image diagnosis system according to a second embodiment;

FIG. 7 is a diagram illustrating setting of a scan condition of a medical image diagnosis system according to a third embodiment;

FIG. 8 is a diagram illustrating setting of a scan condition of a medical image diagnosis system according to a fourth embodiment;

FIG. 9 is a diagram illustrating data processing in the medical image diagnosis system according to the fourth embodiment;

FIG. 10 is a diagram illustrating setting of a scan condition of a medical image diagnosis system according to a fifth embodiment;

FIG. 11 is a diagram illustrating data processing in the medical image diagnosis system according to the fifth embodiment;

FIG. 12 is a diagram illustrating setting of a scan condition of a medical image diagnosis system according to a sixth embodiment; and

FIG. 13 is a diagram illustrating setting of a scan condition of a medical image diagnosis system according to a seventh embodiment.

### DETAILED DESCRIPTION

A medical image diagnosis system according to an embodiment includes a structural image diagnosis apparatus and a nuclear medical image diagnosis apparatus. The system includes a processing circuit that acquires information on a position of a body part of a subject based on a structural image acquired by the structural image diagnosis apparatus and a gantry that scans a nuclear medical image of the subject. The processing circuit controls the gantry based on the information on the position of the body part and a scan condition of the body part.

Hereinafter, exemplary embodiments of the medical image diagnosis system will be explained in detail with reference to the accompanying drawings.

### First Embodiment

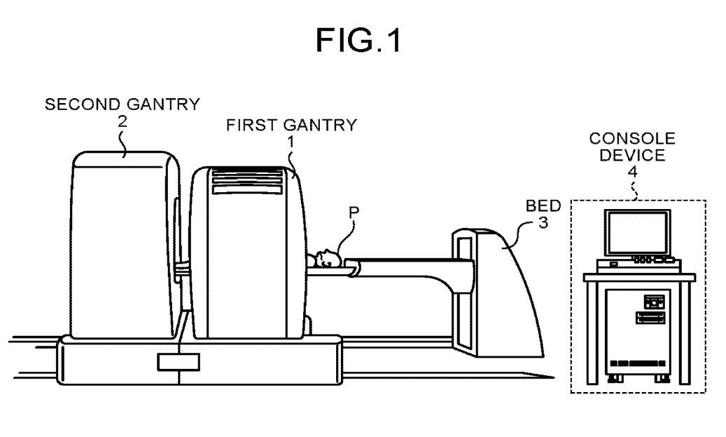

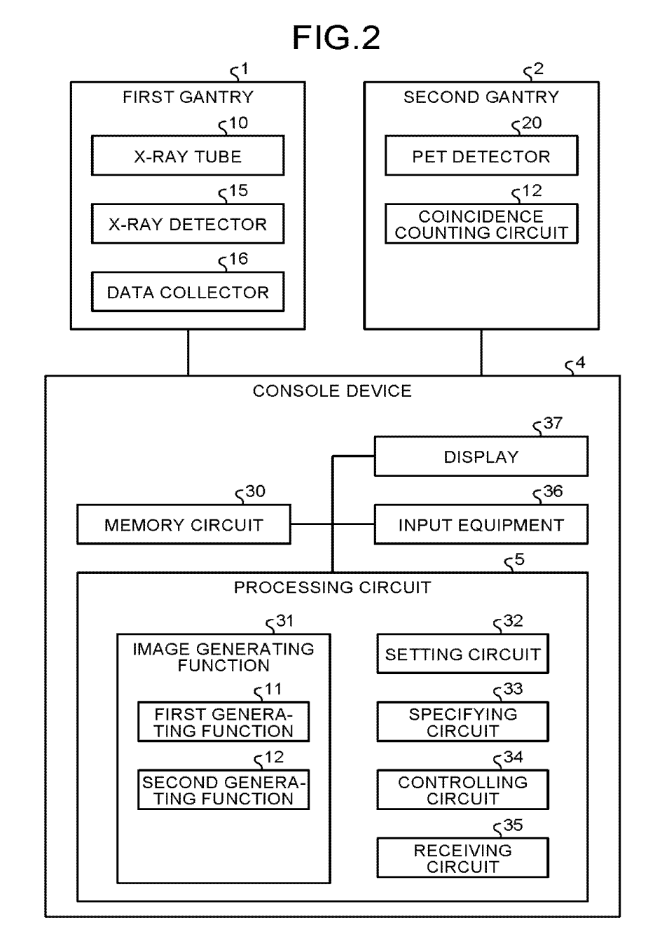

First, an overall configuration of a medical image diagnosis system according to the present embodiment will be explained with reference to FIG. 1 and FIG. 2. FIG. 1 and FIG. 2 are diagrams illustrating an overall configuration of the medical image diagnosis system according to the first embodiment.

As illustrated in FIG. 1, the medical image diagnosis system according to the first embodiment includes a first gantry 1, a second gantry 2, a bed 3, and a console device 4.

The first gantry 1 is, for example, a gantry included in a structural image diagnosis apparatus, and the second gantry 2 is, for example, a gantry included in a functional image diagnosis apparatus. An example of the structural image diagnosis apparatus includes an X-ray Computed Tomography apparatus (X-ray CT apparatus), a Magnetic Resonance Imaging apparatus (MRI apparatus), etc. An example of the functional image diagnosis apparatus includes, for example, a Positron Emission Tomography apparatus (PET apparatus), a Single Photon Emission Computed Tomography apparatus (SPECT apparatus), which are nuclear medical imaging apparatuses. Hereinafter, an example in which the first gantry 1 is a gantry of an X-ray CT scanning apparatus, and the second gantry 2 is a gantry of a PET apparatus will be explained. In this case, the medical image diagnosis system according to the first embodiment is a PET-CT apparatus.

The first gantry 1 detects an X-ray having passed through a subject P to generate X-ray projection data for reconstructing an X-ray CT image and a scanogram.

The first gantry 1 includes an X-ray tube 10, an X-ray detector 15, a data collector 16, etc. as illustrated in FIG. 2. The X-ray tube 10 is a device that generates an X-ray beam, and irradiates the generated X-ray beam to the subject P. The X-ray detector 15 detects the X-ray having passed through the subject P in the position that is opposite to the X-ray tube. Specifically, the X-ray detector 15 is a two-dimensional array type detector that detects data (two-dimensional intensity distribution data) of two-dimensional intensity distribution of the X-ray having passed through the subject P. More specifically, the X-ray detector 15 includes a plurality of X-ray detection element rows, each row being made of X-ray detection elements of a plurality of channels, and the detection element rows are arranged along the body-axial direction of the subject P. The X-ray tube 10 and the X-ray detector 15 are supported by a rotating frame (not illustrated) inside the first gantry 1.

The data collector 16 is, for example, a Data Acquisition System (DAS), and executes amplification processing, A/D conversion processing, etc. for the two-dimensional X-ray intensity distribution data detected by the X-ray detector 15 to generate X-ray projection data. The data collector 16 sends the X-ray projection data to the console device 4 illustrated in FIG. 1. The embodiment is not limited to the case in which the data collector 16 is included in the first gantry 1. Namely, the data collector 16 may be included in the console device 4.

Returning to FIG. 1, the second gantry 2 is a device that detects a pair of gamma rays that is emitted from a body tissue in which positron emitting radionuclides administered to the subject P are introduced, thereby generating projection data of the gamma ray for reconstructing a PET image (gamma ray projection data).

The second gantry includes a PET detector 20, a coincidence counting circuit 12, etc. as illustrated in FIG. 2. The PET detector 20 is a photon counting type detector that detects a gamma ray that is emitted from the subject P. Specifically, a plurality of PET detector modules is configured by being arranged in a manner encompassing the circumference of the subject P in a ring manner.

For example, the PET detector module is an Anger detector that includes a scintillator, a PhotoMultiplier Tube (PMT), and a light guide.

In the scintillator, a plurality of the sodium iodide (NaI), the bismuth germinate (BGO), etc., is arranged two-dimensionally, which convert the gamma ray having been emitted from the subject P and having entered them, to the visible light. The photomultiplier tube is a device that multiplies the visible light output from the scintillator and converts it to an electrical signal, and a plurality of the photomultiplier tubes is densely arranged through the light guide. The light guide is used for transferring the visible light output from the scintillator to the photomultiplier tube, and is made of a plastic material or the like having superior optical transparency.

The photomultiplier tube consists of a photocathode that receives the scintillation light to generate a photoelectron, multistage dynodes that provide the electrical field that accelerates the generated photoelectron, and an anode from which the electron flows off. An electron having been emitted from the photocathode by the photoelectric effect is accelerated toward the dynode to encounter the surface of the dynode, and a plurality of electrons is driven out. This phenomenon is repeated in the multistage dynodes, and thus the number of electrons is multiplied exponentially. The number of electrons in the anode reaches approximately one million. In this example, a gain rate of the photomultiplier tube is one millionfold. Due to the amplification arising from the avalanche phenomenon, the voltage of 1000 V or more is normally applied between the dynode and the anode.

In this way, the PET detector 20 converts a gamma ray to a visible light by the scintillator and further converts the converted visible light to an electrical signal by the photomultiplier tube, and thus the number of the gamma rays having been emitted from the subject P is counted.

The second gantry 2 includes, for example, a coincidence counting circuit 12. The coincidence counting circuit 12 is connected to each of the photomultiplier tubes that are included in a module of PET detectors as a plurality of the PET detectors 20. From the output result of the PET detectors 20, the coincidence counting circuit 12 generates coincidence counting information that is for determining incident directions of a pair of gamma rays having been emitted from the positron. Specifically, the coincidence counting circuit 12 calculates the position of the gravity center from the position and the intensity of an electrical signal of the photomultiplier tube that converts the visible light having been output from the scintillator to the electrical signal at the same timing, and thus determines the incident position of the gamma ray (scintillator position). The coincidence counting circuit 12 calculates (integrates and differentiates) the intensity of the electrical signal that is output from each of the photomultiplier tubes to calculate the energy value of the incident gamma ray.

The coincidence counting circuit 12 searches for the combination in which the incident timings (time) of the gamma rays are in a time window width of a predetermined time, and both of the energy values are in a predetermined energy window width among the output result of the PET detector 20 (Coincidence Finding). For example, the time window width of two nanoseconds and the energy window width of 350 to 550 keV are set as the search condition. The coincidence counting circuit 12 generates coincidence counting information (Coincidence List) while setting the output result of the searched combination as information on which two annihilation photons are coincidence counted. The coincidence counting circuit 12 sends the coincidence counting information to the console device 4 illustrated in FIG. 1 as gamma ray projection data that is for reconstructing the PET image. A line that connects two detection positions in which two annihilation photons are coincidence counted is referred to as the Line of Response (LOR). The coincidence counting information may be generated in the console device 4.

Returning to FIG. 1, the bed 3 is a bed on which the subject P is placed. The bed 3 is sequentially moved to each scan port of the first gantry 1 and the second gantry 2 on the basis of an instruction received via the console device 4, from the operator of the PET-CT apparatus as the medical image diagnosis system.

In other words, by moving the bed 3, the PET-CT apparatus as the medical image diagnosis system executes, first of all, a scan of the X-ray CT image that is a first scan, and subsequently set the condition of the second scan. Subsequently, the medical image system executes a scan of the PET image that is a second scan, on the basis of the set scan condition. For example, the first gantry 1 moves the bed 3 while rotating a rotating frame 15 in the main scan, and performs a helical scan in which a body part of the subject P is helically scanned by the X-ray. The console device 4 generates the X-ray CT image on the basis of the acquired data. The X-ray CT image to be generated may be a 2D image or a 3D image. The first gantry 1 executes a low dose helical scan in a localizer scan. The console device 4 generates a helical scanogram image that is a 3D localizer image on the basis of the acquired data. By moving the bed 3 in the localizer scan while irradiating the X-ray from the X-ray tube in such a state that the rotating frame is fixed, the first gantry 1 may scan the whole body of the subject P along the body-axial direction, and the console device 4 may generate the X-ray CT image (scanogram image) that is a 2D localizer image.

The second gantry 2 moves the bed 3 so that a body part of the subject P to be scanned is inserted into the scan port of the second gantry 2, and thus generates the PET image.

The console device 4 is a device that receives an instruction from the operator to control the scan processing in the medical image diagnosis apparatus.

As illustrated in FIG. 2, the console device 4 includes, for example, a memory circuit 30, a processing circuit 5. The processing circuit 5 includes, for example, an image generating function 31, a setting function 32, a specifying function 33, a controlling function 34, a receiving function 35, an input device 36, and a display 37. An example of the image generating function 31 includes, for example, a first generating function 11 and a second generating function 12.

The setting function 32, the specifying function 33, the receiving function 35, the first generating function 11, and the second generating function 12, which are illustrated in FIG. 2, may be referred to as a setting unit, a specifying unit, a receiving unit, a first generating unit, and a second generating unit, respectively.

In the embodiment illustrated in FIG. 2, each of the processing functions executed in the image generating function 31 (first generating function 11, second generating function 12), the setting function 32, the specifying function 33, the controlling function 34, and the receiving function 35 is stored in the memory circuit 30 in the form of a program that can be executed by a computer. The processing circuit 5 is a processor that reads a program from the memory circuit 30 and executes it to realize a function according to each of the programs. In other words, the processing circuit 5 in a state where it reads each of the programs includes each of the functions in the processing circuit 5 illustrated in FIG. 2. Moreover, in FIG. 2, the processing functions, which is executed in the image generating function 31 (first generating function 11, second generating function 12), the setting function 32, the specifying function 33, the controlling function 34, and the receiving function 35, are explained to be realized in a single processing circuit 5, however, a plurality of independent processors may be combined to configure the processing circuit 5, and each of the processors may execute the program to realize the function.

In other words, each of the aforementioned functions may be configured as a program, and one processing circuit may execute each of the programs, or a specific function may be implemented in a program executing circuit that is dedicated and independent.

“Processor” described in the aforementioned explanation represents, for example, a central processing unit (CPU), a Graphical processing unit (GPU), or a circuit such as an Application Specific Integrated Circuit (ASIC), a Simple Programmable Logic Device (SPLD), a Complex Programmable Logic Device, and a Field Programmable Gate Array (FPGA). The processor reads a program stored in the memory circuit 30 and executes it to realize the function. Moreover, the program may be directly set in the circuit of the processor instead of storing the program in the memory circuit 30. In this case, the processor reads the program that is set in the circuit and executes it to realize the function.

Moreover, the memory circuit 30 memorizes the X-ray projection data sent from the first gantry 1. Specifically, the memory circuit 30 memorizes, by the first generating function 11, the X-ray projection data that is for generating a scanogram and for reconstructing an X-ray CT image.

The processing circuit 5 generates, by the image generating function 31, the scanogram from the X-ray projection data memorized in the memory circuit 30, which is for generating the scanogram. The processing circuit 5 executes, by the first generating function 11, the back projection processing for the X-ray projection data for reconstructing that is memorized in the memory circuit 30 by, for example, a Filtered Back Projection method (FBP method), and thus reconstructs an X-ray CT image.

In other words, the processing circuit 5 generates, by the first generating function 11, a scanogram that is for planning a scan plan in the whole body examination using the PET-CT apparatus to generate a structural image. The processing circuit 5 reconstructs, by the first generating function 11, in a whole-body examination using the PET-CT apparatus, a plurality of X-ray CT images, in which cross-sections perpendicular to the body-axial direction of the subject P are acquired, from the X-ray projection data on the basis of a scan condition (for example, slice width) determined by the scan plan.

For example, the processing circuit 5 reconstructs, by the first generating function 11, volume data or two-dimensional tomographic image data on the basis of the projection data memorized in the memory circuit 30. The volume data and the two-dimensional tomographic image data will be collectively referred to as “image”. A display (not illustrated) is provided in the console device 4 in order to display the image data and an operation screen. The processing circuit 5 receives, by the receiving function 35, an instruction of the operator via the input device 36. The input device 36 is constituted of, a keyboard, a mouse, etc.

The processing circuit 5 extracts, by an anatomical landmark detecting function included in the specifying function 33, a plurality of anatomical feature points from the structural image (helical scanogram image), which are acquired by executing the X-ray CT helical scanning for the subject, on the basis of structural features and the like by the image processing of pattern recognition, etc. The position data of each of the anatomical feature points and the code of each of the anatomical features are saved in the memory circuit 30. The processing circuit 5 may extract, by the anatomical landmark detecting function included in the specifying function 33, a plurality of the anatomical feature points from the image data acquired by a main scan, on the basis of a structural feature and the like by the image processing of pattern recognition, etc.

In other words, the processing circuit 5 specifies, by the specifying function 33, the position of the feature point of the subject P from the structural image, and further specifies the body part of the subject P on the basis of the position of the specified feature point. The processing circuit 5 may specify, by the specifying function 33, the position of the feature point of the subject P from the structural image, and further may specify the body part of the subject P on the basis of the position of the specified feature point, not only in the localizer scan but also in the main scan.

The method, in which the processing circuit 5 specifies the body part of the subject P by the specifying function 33, is not limited to the case in which the body part of the subject P is specified based on the position of the feature point specified by the anatomical landmark detecting function. For example, the processing circuit 5 may specify the aforementioned body part of the subject P by using a method of body part recognition other than the anatomical landmark detection.

Moreover, the processing circuit 5 may receive, by the receiving function 35, an instruction of the operator from an input/output device (not illustrated). For example, the processing circuit 5 accepts, by the receiving function 35, an input of the scan condition that is dependent on the body part of the subject (the scan condition of each body part) in the second scan executed by the second gantry 2, from an input/output device (not illustrated). For example, the processing circuit 5 sets, by the setting function 32, the scan condition of the second scan that is dependent on the body part, for the body part of the subject specified by the specifying function 33. For example, the processing circuit 5 sets, by the setting function 32, the scan condition of the second scan that is dependent on the body part, on the basis of the input of the scan condition accepted by the receiving function 35.

The processing circuit 5 controls, by the controlling function 34, an overall processing of the medical image diagnosis apparatus. Specifically, the processing circuit 5 controls, by the controlling function 34, the first gantry 1 and the second gantry 2 to control the scan by the medical image diagnosis apparatus. The processing circuit 5 controls, by the controlling function 34, the execution of the second generating function 12 by using data that is stored in the memory circuit 30. The processing circuit 5 controls, by the controlling function 34, the execution of the scanogram generating function and the second generating function 12 that use the data stored in the memory circuit 30. The processing circuit 5 controls, by the controlling function 34, an input/output device (not illustrated) to display a Graphical User Interface (GUI) that is for inputting an instruction of the operator, a scanogram, an X-ray CT image, and a PET image. The processing circuit 5 controls, by the controlling function 34, the processing, in which the second gantry 2 generates the functional image on the basis of the second scan, in accordance with a scan condition that is set by the processing circuit 5 using the setting function 32.

The memory circuit 30 memorizes the projection data that is sent from the first gantry 1, and further memorizes the gamma ray projection data that is sent from the coincidence counting circuit included in the second gantry 2. In this case, the processing circuit 5 reconstructs, by the second generating function 12, a PET image from the gamma ray projection data that is stored in the memory circuit 30 by a successive approximation method.

Hereinafter, the successive approximation method executed by the second generating function 12 will be explained. The successive approximation method includes a Maximum Likelihood Expectation Maximization method (MLEM method) or an Ordered Subset MLEM method (OSEM method) whose convergence time is drastically shortened by the improvement of algorithm of the MLEM method.

In the MLEM method, as an initial image, a PET image is reconstructed from the actually collected gamma ray projection data by, for example, a back projection process such as a FBP method. By performing a projection process on the initial image, estimated projection data of the first iteration is generated. By performing a back projection process to the estimated projection data of the first iteration, a reconstructed image of the first iteration is reconstructed. By performing a projection process to the estimated projection data of the first iteration, estimated projection data of the second iteration is generated. By performing a back projection process to the estimated projection data of the second iteration, a reconstructed image of the second iteration is reconstructed. This processing is repeated for the number of operations to be repeated for the successive approximation. Hereinafter, the number of operations to be repeated may be referred to as the iteration number.

Therefore, the estimated projection data whose ratio to actually collected projection data has converged to approximately “1”, is generated. The reconstructed image generated from the converged estimated projection data by the back projection process is a PET image that represents the stochastically most probable amassment distribution of the positron emitting radionuclide.

The OSEM method is a method in which the gamma ray projection data is divided into several subsets and the aforementioned successive approximation is executed for each of the subsets to correct the image. In other words, the OSEM method whose number of subsets is “1” is the MLEM method.

The total number of calculations in the second generating function 12 depends on the number of iterations in the case of the MLEM method. The total number of calculations in the second generating function 12 depends on the number that is the result of multiplication of the number of subsets by the number of iterations in the case of OSEM method.

Hereinafter, a case will be explained in which the processing circuit 5 reconstructs, by the OSEM method, a PET image in the second generating function 12. The embodiment is also applicable to the case in which the processing circuit 5 reconstructs, in the second generating function 12, a PET image by the MLEM method.

The processing circuit 5 included in the console device 4 of the medical image diagnosis system according to the first embodiment includes the first generating function 11, the specifying function 33, a setting function 22, and the second generating function 12. The processing circuit 5 generates, by the first generating function 11, a structural image on the basis of the first scan for the subject. The processing circuit 5 specifies, by the specifying function 33, the position of the feature point of the subject from the structural image, and specifies a body part of the subject on the basis of the specified position of the feature point. The processing circuit 5 sets, by the setting function 22, a scan condition of the second scan that is dependent on the body part (a scan condition of each body part), for the body part specified by the specifying function 33. The processing circuit 5 generates, by the second generating function, a functional image (a nuclear medical image) on the basis of the second scan that is executed in accordance with the scan condition that is dependent on the body part.

In other words, the medical image diagnosis system according to the first embodiment is a medical image diagnosis system that includes, for example, a structural image diagnosis apparatus (for example, first gantry 1) and a nuclear medical image diagnosis apparatus (for example, second gantry). The medical image diagnosis system includes the processing circuit 5 and the gantry (for example, second gantry 2). The processing circuit 5 acquires information on the body part of the subject P on the basis of the structural image that is acquired by the structural image diagnosis apparatus. The gantry acquires the nuclear medical image of the subject. The processing circuit 5 controls the gantry on the basis of the acquired information on the body part of the subject P and the scan condition of each body part.

Moreover, the processing circuit 5 may further include the receiving function 35. In this case, the processing circuit 5 accepts the input of the scan condition that is dependent on the body part by the receiving function 35. The processing circuit 5 sets, by the setting function 22, the scan condition of the second scan that is dependent on the body part, on the basis of the input of the scan condition accepted by the receiving function 35.

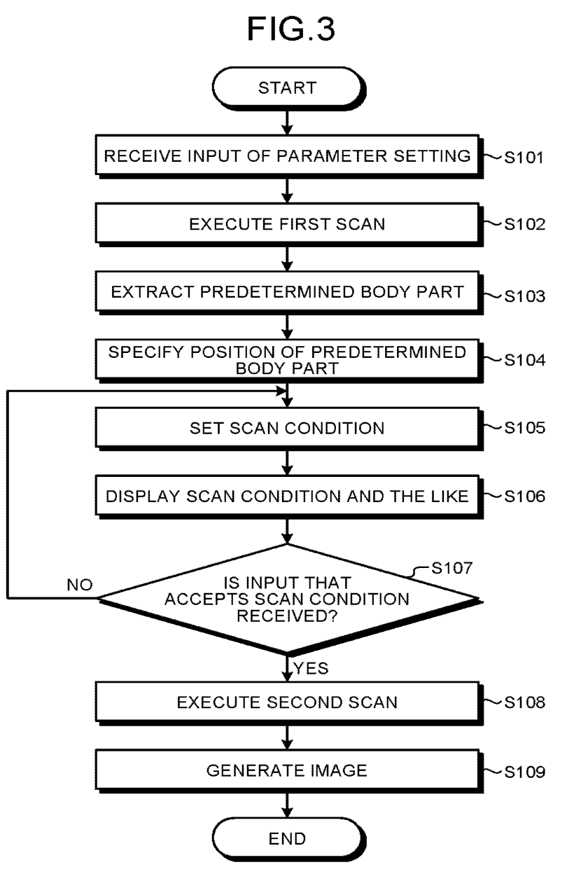

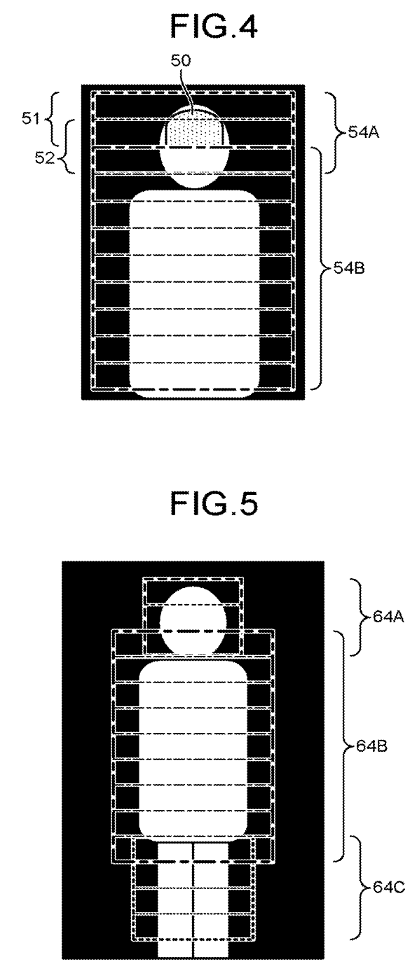

These points will be explained with reference to FIGS. 3 and 4. FIG. 3 is a flowchart illustrating a processing procedure of the medical image diagnosis system according to the first embodiment. The processing circuit 5 calls a predetermined program according to various functions from the memory circuit 30, and further executes them to realize various functions. FIG. 4 is a diagram illustrating setting of a scan condition of the medical image diagnosis system according to the first embodiment. Hereinafter, each step of the flowchart illustrated in FIG. 3 will be explained with proper reference to FIG. 4.

The processing circuit 5 accepts, by the receiving function 35, an input of a scan condition that is dependent on the body part of the subject (a scan condition of each body part) in a second scan. The second scan is, for example, the PET scan using the second gantry 2. The processing circuit 5 accepts, by the receiving function 35, an input of parameter setting from the operator (Step S101). The aforementioned parameter is, for example, a specific body part, the acquisition time of the specific body part, and the acquisition time of the whole-body scan (more specifically, scan of body parts other than the specific body part). In other words, the processing circuit 5 accepts, by the receiving function 35, an input of a scan time duration or a scan speed in a scanning range as the scan condition that is dependent on the body part of the subject (a scan condition of each body part). For example, the processing circuit 5 accepts, by the receiving function 35, an input of the acquisition time “5 minutes per single position”, for a body part “head part” from the operator as a scan condition that is dependent on the body part. The processing circuit 5 accepts, by the receiving function 35, an input of the acquisition time “2 minutes per single position”, for the body part “whole body (body parts other than head part)” as a scan condition that is dependent on the body part. The “single position” indicates, for example, in such a case that the scan is executed in a step-and-shoot method using the PET apparatus in the second scan, a scanning range executed at one step.

As another example, the processing circuit 5 may, by the receiving function 35, instead of accepting an input of the the acquisition time per single position, accept an input of the acquisition time needed for scanning all of the predetermined body parts from the operator. For example, the processing circuit 5 may accept, by the receiving function 35, an input of the acquisition time “15 minutes in all” for “head part” as the scan condition that is dependent on the body part, and further accept an input of the acquisition time “20 minutes in all” for “whole body (body parts other than head part)” as the scan condition that is dependent on the body part.

When the processing circuit 5 accepts, by the receiving function 35, an input of the parameter setting from the operator, the first gantry 1 executes the first scan (Step S102). The first scan is, for example, the X-ray CT scan using the X-ray CT scanning apparatus. For example, the first gantry 1 executes an X-ray CT scan by the low dose helical scan.

The processing circuit 5 generates, by the first generating function 11, a structural image on the basis of the first scan for the subject.

The processing circuit 5 specifies, by the specifying function 33, the position of a feature point of the subject from the generated structural image. In other words, the processing circuit 5 extracts a predetermined body part (Step S103). For example, processing software that configures the processing circuit 5 automatically extracts a predetermined body part from the acquired low dose helical scan image.

Subsequently, the processing circuit 5 specifies, by the specifying function 33, a predetermined body part of the subject on the basis of the specified position of the feature point. Alternatively, the processing circuit 5 specifies, by the specifying function 33, a plurality of body parts of the subject on the basis of the specified position of the feature point. In other words, the processing circuit 5 specifies, by the specifying function 33, the position of a predetermined body part (or a plurality of body parts) (Step S104).

In FIG. 4, the body parts specified in this way by the specifying function 33 of the processing circuit 5 is illustrated in which they are overlapped with the scanning range of the second scan. In this example, the processing circuit 5 specifies, by the specifying function 33, a body part “head part 50A” as a scanning range 54A. The processing circuit 5 specifies, by the specifying function 33, a body part “whole body (body parts other than head part)” as a scanning range 54B.

The processing circuit 5 sets, by the setting function 32, a scan condition of the second scan that is dependent on the body part, for the body part specified by the specifying function 33. Alternatively, the processing circuit 5 sets, by the setting function 32, for each of the specified body parts, a scan condition of the second scan that is dependent on the each body part. For example, the processing circuit 5 sets, by the setting function 32, a scan condition of the second scan that is dependent on a body part, on the basis of the input of scan condition accepted by the receiving function 35 (Step S105). For example, the processing circuit 5 sets, by the setting function 32, acquisition time (scan time duration or scan speed).

For example, the processing circuit 5 sets, by the setting function 32, a scan condition “5 minutes per 1 position” that is dependent on the body part for the scanning range 54A specified as a body part “head part”. Moreover, the processing circuit 5 sets, by the setting function 32, a scan condition “2 minutes per 1 position” that is dependent on the body part for the scanning range 54B specified as a body part “whole body (body parts other than head part)”.

Instead of setting the acquisition time per single position, the processing circuit 5 may set, by the setting function 32, acquisition time needed for scan all of the predetermined body parts. For example, the processing circuit 5 may set, by the setting function 32, a scan condition “15 minutes in all” that is dependent on the body part, for the scanning range 54A specified as a body part “head part”, and further may set a scan condition “20 minutes in all” that is dependent on a body part, for the scanning range 54B specified as a body part “whole body (body parts other than head part)”.

The processing circuit 5 may set, if necessary, other parameters by the setting function 32.

It is noted that each of the areas 51 and 52 represents the first scan area and the second scan area in the scan of the body part “head part” in the second scan. In this way, the second gantry 2 executes the second scan while overlapping the scan position at each “position”.

When setting scan conditions such as the acquisition time or other parameters, the processing circuit 5 displays the set scan condition and the like on a display device such as a display (Step S106). For example, the processing circuit 5 displays, on a display, a message that indicates the fact that the scan condition “5 minutes per 1 position” is set for a body part “head part”, and a scanning condition “2 minutes per single position” is set for a body part “whole body (body parts other than head part)”. For example, the processing circuit 5 displays, on the display, the scanning range 54A specified as the body part “head part” and the scanning range 54B specified as the body part “whole body (body parts other than head part)”, overlapping them with the structural image generated by the first scan. Moreover, the processing circuit 5 also displays, on the display, a message that indicates the fact that, in the structural image, the scan condition “5 minutes per 1 position” is set for the body part “head part”, and the scanning range “2 minutes per 1 position” is set for the body part “whole body (body parts other than head part)”.

The processing circuit 5 accepts an input from the operator via the input device 36. The processing circuit 5 determines, by the receiving function 35, on the basis of the input from the operator, whether or not an input of an approval of the scan condition is accepted (Step S107). When the operator does not accept the scan condition, the processing circuit 5 determines, by the receiving function 35, that an input of an approval of the scan condition is not accepted (Step S107: No), and repeats the processing similar to Step S101 to accept a re-input of a parameter setting from the operator. Subsequently, the processing circuit 5 returns to Step S105, and then sets, by the setting function 32, the scan condition again on the basis of the parameter re-input from the operator.

When the operator accepts the scan condition, the processing circuit 5 determines, by the receiving function 35, that the input of the approval of the scan condition is accepted (Step S107: Yes), the second gantry 2 executes the second scan that is for generating a functional image in accordance with the scan condition that is dependent on the body part set by the setting function 32, by the processing circuit 5. Alternatively, the second gantry 2 executes the second scan that is for generating a functional image, in accordance with the scan condition that is dependent on each of the body parts set by the setting function 32, by the processing circuit 5 (Step S108).

When the second scan is terminated, the processing circuit 5 generates, by the second generating function 12, a functional image on the basis of the second scan that is executed in accordance with the scan condition that is dependent on the body part (Step S109), or generates the functional image on the basis of the second scan that is executed in accordance with the scan condition that is dependent on each of the body parts and the processing is terminated.

In this way, the processing circuit 5 according to the first embodiment specifies, by the specifying function 33, the position of a feature point of the subject from a structural image, and further specifies a body part of the subject on the basis of the specified position of the feature point. The processing circuit 5 sets, by the setting function 32, a scan condition of the second scan that is dependent on the body part, for the specified body part. The processing circuit 5 generates, by the second generating function 12, a functional image on the basis of the second scan that is executed in accordance with the scan condition that is dependent on the body part. The input of a scan time duration or a scan speed is accepted as the scan condition that is dependent on the body part.

Therefore, by employing the medical image diagnosis system according to the first embodiment, the burden of the operator can be lessened. By employing different scan speed between the body part in which high-precision scan is needed for a diagnosis and the body part in which high-precision scan is not needed, an image with good quality can be acquired in a limited scan time duration of a patient. By employing the medical image diagnosis system according to the first embodiment, the manual setting of the scanning range by an operator is not necessary. As a result, an image with good quality can be acquired while lessening the burden of the operator.

### Second Embodiment

In the first embodiment, the case is explained in which the input of a scan time duration or a scan speed is accepted as the scan condition that is dependent on the body part. In the second embodiment, the size of a Field Of View (FOV) is set as the scan condition that is dependent on a body part.

Thus, as the setting of unnecessarily large size of FOV for the body part is not required, the error arising from events corresponding to areas irrelevant to the scan (for example, scattered component of background) can be reduced, and thus the image quality of the functional image can be improved.

The processing circuit 5 included in the medical image diagnosis system according to the second embodiment sets, by the setting function 32, the size of the FOV.

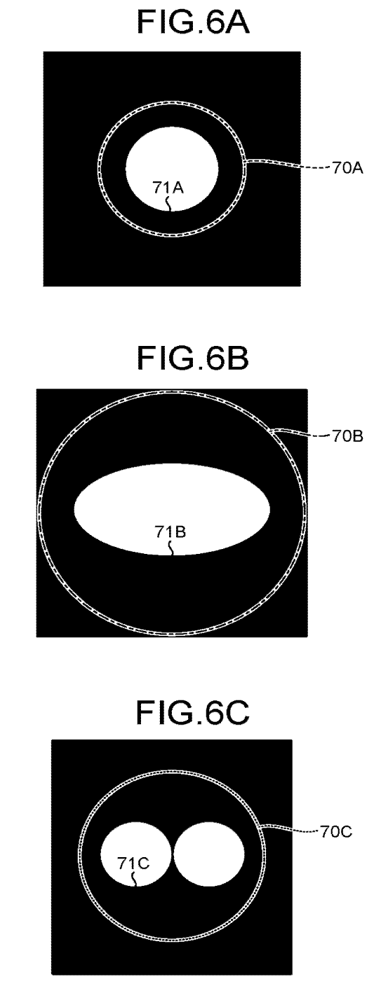

The processing executed by the medical image diagnosis system according to the second embodiment will be explained with reference to FIG. 3, FIG. 5, and FIGS. 6A to 6C. FIG. 5 and FIGS. 6A to 6C are diagrams illustrating setting of a scan condition of the medical image diagnosis system according to the second embodiment.

In the second embodiment, similarly to the first embodiment, the processing illustrated in the flowchart in FIG. 3 is executed, however, the processing of Step S105 is different from that of the first embodiment. Hereinafter, detailed explanation will be omitted with regard to execution of the processing similar to the first embodiment.

As illustrated in FIG. 5, in the second embodiment, the processing similar to that of the first embodiment is executed, and thus the processing circuit 5 specifies, by the specifying function 33, a predetermined body part of the subject on the basis of the specified position of the feature point in Step S104. In the example illustrated in FIG. 5, the processing circuit 5 specifies, by the specifying function 33, a body part “head part” as a scanning range 64A. The processing circuit 5 specifies, by the specifying function 33, a body part “shoulder to lower abdomen part” as a scanning range 64B. The processing circuit 5 specifies, by the specifying function 33, a body part “lower extremity” as a scanning range 64C.

In the second embodiment, the processing circuit 5 sets, by the setting function 32, the size of FOV in Step S105. In FIG. 6A, an axial tomogram image for a body part “head part” is illustrated, and the boundary of an area 71A corresponds to a body surface of the subject. Similarly, in FIG. 6B, an axial tomogram image for a body part “shoulder to lower abdomen part” is illustrated, and the boundary of an area 71B corresponds to a body surface of the subject. Similarly, in FIG. 6C, an axial tomogram image for a body part “lower extremity” is illustrated, and the boundary of an area 71C corresponds to a body surface of the subject.

The processing circuit 5 sets, by the setting function 32, the area 71A illustrated in FIG. 6A as an FOV for the scanning range 64A specified as the body part “head part”. Specifically, the processing circuit 5 sets, by the setting function 32, the area including the whole body surface 70A of the subject as the area 71A. Similarly, the processing circuit 5 sets, by the setting function 32, the area 71B illustrated in FIG. 6B as an FOV for the scanning range 64B specified as the body part “shoulder to lower abdomen part”. The processing circuit 5 sets, by the setting function 32, the area 71C illustrated in FIG. 6C as an FOV for the scanning range 64C specified as the body part “lower extremity”. The processing circuit 5 executes the processing similar to that of the first embodiment on the basis of the scan condition that is set in this way.

In this way, the processing circuit 5 according to the second embodiment sets, by the setting function 32, the size of FOV. This is because of the following reason. In PET, the detector detects one pair of γ-rays that are generated by annihilation. If FOV is unnecessarily large for the body part, the detector detects a signal (scattered component of background) of the γ-ray generated in the position irrelevant to the subject. As a result, the noise increases, and thus the image quality of an acquired functional image decreases. Therefore, it is desirable that the size of FOV be not unnecessarily large compared to the body part of the subject. In the second embodiment, by the processing circuit 5 automatically setting the size of the FOV by the setting function 32, the size of FOV need not be unnecessarily large for the body part. As a result, the error arising from an event corresponding to the areas irrelevant to the scan can be reduced, and thus the image quality of the functional image can be improved. Although this operation may be manually executed by the operator, if the operator manually executes the operation that sets the size of FOV, the burden of the operator is heavy. By employing the medical image diagnosis system according to the second embodiment, one can maintain the image quality of a functional image while lessening the burden of the operator at the same time.

### Third Embodiment

In the third embodiment, as the scan condition that is dependent on the body part, the input of the size of an overlap area in each scan is set. Therefore, shortening a scan time duration while maintaining the image quality (signal-to-noise ratio: S/N ratio) of the functional image can be realized while lessening the burden of the operator.

In the third embodiment, in a case in which the second scan of a predetermined body part is divided into a plurality of times, the processing circuit 5 accepts, by the receiving function 35, an input of the size of the overlap area in each scan, as the scan condition that is dependent on the body part. The processing circuit 5 sets, by the setting function 32, a scan condition that is dependent on the body part, in the second scan, on the basis of the accepted input of the scan condition.

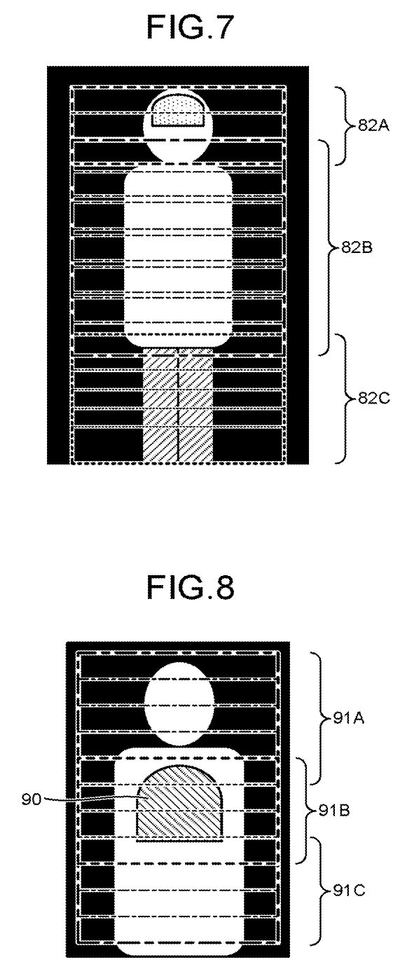

The processing executed by the medical image diagnosis system according to the third embodiment will be explained with reference to FIGS. 3 and 7. FIG. 7 is a diagram illustrating setting of a scan condition of the medical image diagnosis system according to the third embodiment.

In the third embodiment, although the processing illustrated in the flowchart in FIG. 3 is executed similarly to the first embodiment, the processing of Steps S101 and S105 is different from those of the first embodiment. Hereinafter, detailed explanation will be omitted with regard to execution of the processing similar to the first embodiment.

As described above, the second gantry 2 usually executes the second scan while overlapping scan location in each position. The processing circuit 5 accepts an input of the size of overlap area as the scan condition. Specifically, in Step S101, in a case in which the second scan for a predetermined body part is divided into a plurality of times of scans and the plurality of times of scans are executed sequentially, as the scan condition that is dependent on the body part of the subject (the scan condition of each of the plurality of body parts), the processing circuit 5 accepts, by the receiving function 35, an input of the size of the overlap area in each scan as the scan condition that is dependent on the body part. For example, the processing circuit 5 accepts, by the receiving function 35, for a body part “head part”, the input of the size of overlap area “50%” as the scan condition that is dependent on the body part of the subject. The processing circuit 5 accepts, by the receiving function 35, for a body part “lower extremity”, an input of the size of the overlap area “30%” as the scan condition that is dependent on the body part of the subject. The processing circuit 5 accepts, by the receiving function 35, for a body part “others”, an input of the size of the overlap area “40%” as the scan condition that is dependent on the body part of the subject.

As illustrated in FIG. 7, in the third embodiment, by executing the processing similar to that of the first embodiment, the processing circuit 5 specifies, by the specifying function 33, a predetermined body part of the subject on the basis of a specified position of the feature point in Step S104. In the example illustrated in FIG. 7, the processing circuit 5 specifies, by the specifying function 33, a body part “head part” as the scanning range 82A. The processing circuit 5 specifies, by the specifying function 33, a body part “lower extremity” as the scanning range 82C. The processing circuit 5 specifies, by the specifying function 33, a body part “others” as the scanning range 82B.

In the third embodiment, in Step S105, the processing circuit 5 sets, by the setting function 32, the size of overlap area of each scan.

The processing circuit 5 sets, by the setting function 32, “50%” as the size of overlap area of scan in the scanning range 82A specified as the body part “head part”. Similarly, the processing circuit 5 sets, by the setting function 32, “30%” as the size of overlap area of scan in the scanning range 82B specified as the body part “lower extremity”. Similarly, the processing circuit 5 sets, by the setting function 32, “40%” as the size of overlap area of scan in the scanning range 82C specified as the body part “others”. The processing circuit 5 executes the processing similar to that of the first embodiment on the basis of the scan condition that is set in this way.

In this way, the processing circuit 5 according to the third embodiment sets, by the setting function 32, the size of overlap area of each scan. This is because of the following reason. With regard to PET, in a case in which the scan is executed in the step-and-shoot method, since γ-ray is detected at edge portion of a detector in both ends of each of the positions, the detection efficiency decreases compared to that of the center of each of the positions. Therefore, in order to compensate the effect, an overlap area at each scan is being set to compensate the data. The larger the size of overlap area is, the more the accuracy (S/N ratio) of the data increases. However, the larger the overlap area is, the more the total scan time duration increases. Since the accuracy of the data required in the end is different from body part to body part, by setting the size of overlap area for each of the body parts, the precision of the data in the relevant body part can be maintained while shortening the total scan time duration. Although this operation may be manually executed by an operator, if the operation of the setting of the size of the overlap area is manually executed by the operator, the burden of the operator is heavy. By employing the medical image diagnosis system according to the third embodiment, shortening the scan time duration while maintaining the image quality (S/N ratio) of the functional image can be realized while lessening the burden of the operator.

### Fourth Embodiment

The processing circuit 5 according to the fourth embodiment sets a scan condition of a synchronous scan such as a respiratory synchronous scan or an electrocardiographic synchronous scan.

The processing circuit 5 according to the fourth embodiment accepts, by the receiving function 35, an input of a scan condition of a synchronous scan as the scan condition that is dependent on the body part (the scan condition of each body part). The processing circuit 5 sets, by the setting function 32, the scan condition of the second scan that is dependent on a body part, on the basis of the accepted input of the scan condition.

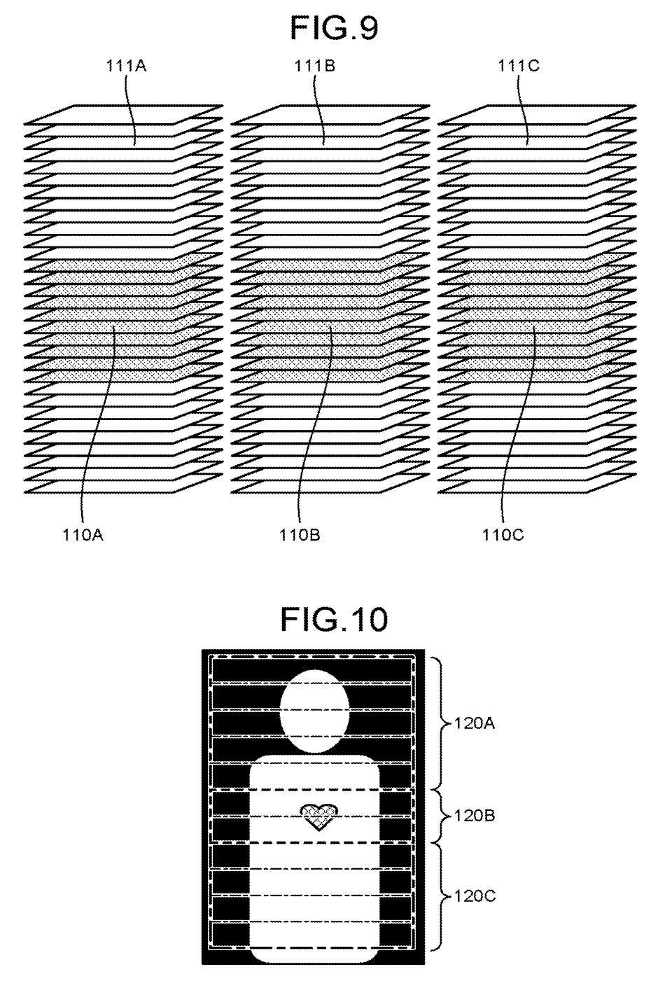

The processing executed by the medical image diagnosis system according to the fourth embodiment will be explained with reference to FIGS. 3, 8, and 9. FIG. 8 is a diagram illustrating setting of the scan condition of the medical image diagnosis system according to the fourth embodiment. FIG. 9 is a diagram illustrating data processing in the medical image diagnosis system according to the fourth embodiment.

In the fourth embodiment, similarly to the first embodiment, the processing illustrated in the flowchart in FIG. 3 is executed. Hereinafter, detailed explanation will be omitted with regard to execution of the processing similar to the first embodiment.

In Step S101, the processing circuit 5 accepts, by the receiving function 35, an input of the scan condition of a synchronous scan as the scan condition that is dependent on the body part (the scan condition of each body part). The processing circuit 5 accepts, by the receiving function 35, the input of “execution of respiratory synchronization” and “parameter regarding respiratory synchronization” for a body part “chest part 90” illustrated in FIG. 8 as the scan condition that is dependent on the body part of the subject. As another example, the processing circuit 5 accepts, by the receiving function 35, for a body part “heart”, an input of “execution of electrocardiographic synchronization” and “parameter regarding electrocardiographic synchronization” as the scan condition that is dependent on the body part of the subject. As another example, the processing circuit 5 accepts, by the receiving function 35, for a predetermined body part, an input of “execution of synchronous scan that is a combination of respiratory synchronization and electrocardiographic synchronization” and “predetermined parameter” as the scan condition that is dependent on the body part of the subject. Moreover, the processing circuit 5 accepts, by the receiving function 35, an input of a predetermined condition of “head part”, “others”, etc., which are illustrated in FIG. 8, as the scan condition that is dependent on the body part of the subject.

A synchronous scan is a scan in which the scan is executed in synchronization with an event repeating and continuing at a predetermined interval, including a respiratory synchronization in which the scan is executed in synchronization with the respiratory phase, and an electrocardiographic synchronous scan in which the scan is executed in synchronization with the electrocardiographic phase. When the respiratory synchronous scan is executed, the second gantry 2 executes scan at a plurality of times corresponding to respiratory phases, and collects the data at each of the times. The processing circuit 5 organizes, by the second generating function, for each of the respiratory phases, the data at each of the times corresponding to each of the respiratory phases, thereby generating a functional image for each of the respiratory phases (expansion phase, contraction phase, etc.). An example of the parameter regarding the respiratory synchronization includes the number of scans of the respiratory synchronous scan, the scan starting time, the scan ending time, the scan time duration, the scan interval, etc. When the electrocardiographic synchronous scan is executed, the second gantry 2 executes scan at a plurality of times, associating them with electrocardiographic phases, and collects the data at each of the plurality of times. The processing circuit 5 organizes, by the second generating function, for each of the electrocardiographic phases, the data at each of the times associated with electrocardiographic phases, thereby generating a functional image for each of the electrocardiographic phases (systolic phase, diastole phase). An example of the parameter regarding the electrocardiographic synchronization includes the number of scans of the electrocardiographic synchronous scan, the scan starting time, the scan ending time, the scan time duration, the scan interval, etc.

As illustrated in FIG. 8, in the fourth embodiment, the processing similar to that of the first embodiment is executed, and thus the processing circuit 5 specifies, by the specifying function 33, a predetermined body part of the subject on the basis of the specified position of the feature point in Step S104. In the example illustrated in FIG. 8, the processing circuit 5 specifies, by the specifying function 33, a body part “head part” as a scanning range 91A. The processing circuit 5 specifies, by the specifying function 33, a body part “chest part” as a scanning range 91B. The processing circuit 5 specifies, by the specifying function 33, a body part “others” as the scanning range 82B.

In the fourth embodiment, the processing circuit 5 sets, by the setting function 32, a scan condition of the synchronous scan as the scan condition that is dependent on the body part (the scan condition of each body part) in Step S105. The processing circuit 5 sets, by the setting function 32, “execution of respiratory synchronous scan” and “parameter regarding respiratory synchronous scan” as the scan condition in the scanning range 91B specified as the body part “chest part”. The processing circuit 5 executes, by the setting function 32, the setting similar to that of the first embodiment with regard to a body part other than the body part “chest part”.

In Step S108, the second gantry 2 executes the second scan in accordance with the scan condition that is dependent on the body part set by the setting function 32, by the processing circuit 5.

For example, this situation is illustrated in FIG. 9. The second gantry 2 executes the scan at the time pertinent to the first respiratory phase to collect data 110A and 111A. The second gantry 2 executes the scan at the time pertinent to the second respiratory phase to collect data 110B and 111B. The second gantry 2 executes the scan at the time pertinent to the third respiratory phase to collect data 110C and 111C.

The data 110A, 110B, and 110C are pertinent to the body part “chest part” that is the target of the synchronous scan. The data 111A, 111B, and 111C are related to the body part other than “chest part”.

In Step S109, when the second scan is terminated, the processing circuit 5 generates, by the second generating function 12, a functional image on the basis of the second scan that is executed in accordance with the scan condition that is dependent on the body part. Specifically, the processing circuit 5 merges, by the second generating function 12, the data for each phase pertinent to the body part in which the synchronous scan is executed, and, on the basis of the result, generates an image of each phase. On the other hand, the processing circuit 5 merges, by the second generating function 12, the data of all of the phases pertinent to the body part in which the synchronous scan is not executed, and, on the basis of the result, generates one image.

Specifically, with regard to the body part “chest part”, the processing circuit 5 merges, by the second generating function 12, the data 110A according to the first respiratory phase, the data 110B according to the second respiratory phase, and the data 110C according to the third respiratory phase to generate the data of each of the respiratory phases, and, on the basis of the result, generates the image of each of the respiratory phases. On the other hand, with regard to the body part other than the body part “chest part”, the processing circuit 5 merges, by the second generating function 12, the data 111A, 111B, and 111C to generate one data, and, on the basis of the result, generates an image.

In this way, the processing circuit 5 according to the fourth embodiment sets, by the setting function 32, a scan condition of a synchronous scan as the scan condition that is dependent on the body part. This is because of the following reason. As a body part such as a chest part or a heart is non-static, it is desirable that a particular processing such as the synchronous scan be executed, unlike other body parts. However, manually executing such a parameter setting is bothersome for the operator. By employing the medical image diagnosis system according to the fourth embodiment, the processing circuit 5 sets a scan condition of a synchronous scan, and thus the burden of an operator can be lessened.

### Fifth Embodiment

In the fifth embodiment, the processing circuit 5 sets a scan condition of a dynamic scan.

The processing circuit 5 according to the fifth embodiment accepts, by the receiving function 35, an input of a scan condition of a dynamic scan as the scan condition that is dependent on the body part (the scan condition of each body part). The processing circuit 5 sets, by the setting function 32, a scan condition of the second scan that is dependent on the body part, on the basis of the accepted input of the scan condition.

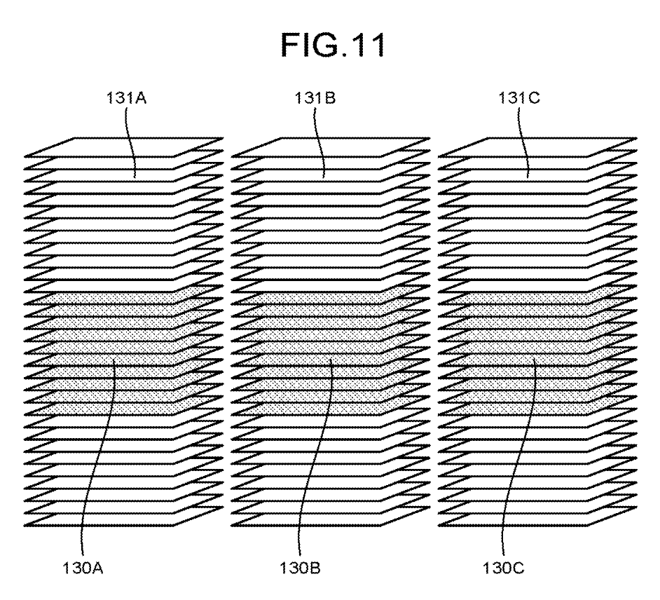

The processing executed by the medical image diagnosis system according to the fifth embodiment will be explained with reference to FIGS. 3, 10, and 11. FIG. 10 is a diagram illustrating setting of a scan condition of the medical image diagnosis system according to the fifth embodiment. FIG. 11 is a diagram illustrating data processing in the medical image diagnosis system according to the fifth embodiment.

In the fifth embodiment, similarly to the first embodiment, the processing illustrated in the flowchart in FIG. 3 is executed. Hereinafter, detailed explanation will be omitted with regard to execution of the processing similar to the first embodiment.

In Step S101, the processing circuit 5 accepts, by the receiving function 35, an input of a scan condition of a dynamic scan as the scan condition that is dependent on the body part (the scan condition of each body part). The processing circuit 5 accepts, by the receiving function 35, an input of “execution of dynamic scan” and “parameter regarding dynamics” for the body part “heart” illustrated in FIG. 10 as a scan condition that is dependent on the body part of the subject. Moreover, the processing circuit 5 accepts, by the receiving function 35, an input of a predetermined condition of a body part “head part”, a body part “others”, etc., which are illustrated in FIG. 8, as the scan condition that is dependent of the body part of the subject.

A dynamic scan is a scan capable of observing, for example, the temporal change in a predetermined body part, by scanning along a time series (time phases). The dynamic scan and the synchronous scan have in common in that the temporal change of a predetermined body part can be observed. In a synchronous scan, motion at a predetermined phase of a periodically repeated motion such as aspiration or heartbeat is focused. On the other hand, a dynamic scan is, for example, a scan that focuses attention on transient characteristics when a contrast agent is injected. A dynamic scan is executed for observing, for example, the intralesional blood vessel dynamics. For example, in the case of a normal scan, a contrast agent is injected at a predetermined injection speed, and the scan is started when the concentration of the contrast agent in the blood reaches equilibrium. As a result, the scan in the equilibrium phase can be executed. On the other hand, in a dynamic scan, for example, the injection speed of the contrast agent is made higher than that of a normal scan to inject the contrast agent in shorter time duration, and a scan is executed before the contrast agent concentration of the blood reaches equilibrium, thereby observing the transient characteristics (artery layer).

An example of the parameter regarding the dynamic scan includes the number of scans of the dynamic scan, the scan starting time, the scan ending time, the scan time duration, the scan interval, etc.

As illustrated in FIG. 10, in the fifth embodiment, by executing the processing similar to that of the first embodiment, the processing circuit 5 specifies, by the specifying function 33, a predetermined body part of the subject on the basis of the specified position of a feature point in Step S104. In the example illustrated in FIG. 10, the processing circuit 5 specifies, by the specifying function 33, a body part “head part” as the scanning range 120A. The processing circuit 5 specifies, by the specifying function 33, a body part “heart” as the scanning range 120B. The processing circuit 5 specifies, by the specifying function 33, a body part “lower extremity” as the scanning range 120C.

In the fifth embodiment, the processing circuit 5 sets, by the setting function 32, a scan condition of a dynamic scan as the scan condition that is dependent on the body part (the scan condition of each body part) in Step S105. The processing circuit 5 sets, by the setting function 32, “execution of dynamic scan” and “parameter regarding dynamic scan” as the scan condition in the scanning range 120B specified as the body part “heart”. The processing circuit 5 executes, by the setting function 32, the setting similar to that of the first embodiment with regard to the body part other than the body part “heart”.

In Step S108, the second gantry 2 executes the second scan in accordance with the scan condition that is dependent on the body part set by the setting function 32, by the processing circuit 5.

For example, this situation is illustrated in FIG. 11. The second gantry 2 executes the scan at the time pertinent to the first time phase, and collects data 130A and 131A. The second gantry 2 executes the scan at the time according to the second time phase, and collects data 130B and 131B. The second gantry 2 executes the scan at the time according to the third time phase, and collects data 130C and 131C.

The data 130A, 130B, and 130C are pertinent to the body part “heart” that is the target of the dynamic scan. The data 131A, 131B, and 131C are pertinent to the body part other than the body part “heart”.

In Step S109, when the second scan is terminated, the processing circuit 5 generates, by the second generating function 12, a functional image on the basis of the second scan that is executed in accordance with a scan condition that is dependent on the body part. Specifically, the processing circuit 5 merges, by the second generating function 12, the data pertinent to the body part in which the dynamic scan is executed, and, on the basis of the result, generates a time series image. On the other hand, the processing circuit 5 merges, by the second generating function 12, the data of all of the time phases pertinent to the body part in which the dynamic scan is not executed, and, on the basis of the result, generates one image.

Specifically, with regard to the body part “heart”, the processing circuit 5 merges, by the second generating function 12, the data 130A pertinent to the first time phase, the data 130B pertinent to the second time phase, and the data 130C pertinent to the third time phase to generate the data of each of the time phases, and, on the basis of the result, generates the time series image. On the other hand, with regard to the body part other than the body part “heart”, the processing circuit 5 merges, by the second generating function 12, the data 131A, 131B, and 131C to generate one data, and, on the basis of the result, generates an image.

In this way, the processing circuit 5 according to the fifth embodiment sets, by the setting function 32, a scan condition of a dynamic scan as the scan condition that is dependent on the body part. The body part in which the dynamic scan is executed needs a parameter setting that is different from that of a body part in which a usual scan is executed. However, manually executing such a parameter setting is bothersome for the operator. By employing the medical image diagnosis system according to the fifth embodiment, the processing circuit 5 sets the scan condition of a dynamic scan, and thus the burden of the operator can be lessened.

### Sixth Embodiment

So far, a case is explained in which the second gantry 2 executes a scan in the step-and-shoot method. However, the embodiment is not limited to this example. In the sixth embodiment, the second scan is a continuous scan that is executed while continuously moving a body part of the subject to be scanned.

The continuous scan method is a scan method in which, instead of executing the scan in the step-and-shoot method, the scan is executed while continuously moving a body part of the subject to be scanned. As the body part to be scanned changes discretely in the step-and-shoot method, spatial fluctuation of a precision of a generated image arises. The medical image diagnosis system according to the sixth embodiment executes a scan while continuously moving the body part of the subject to be scanned, and thus spatial fluctuation of the precision of the generated image can be reduced.

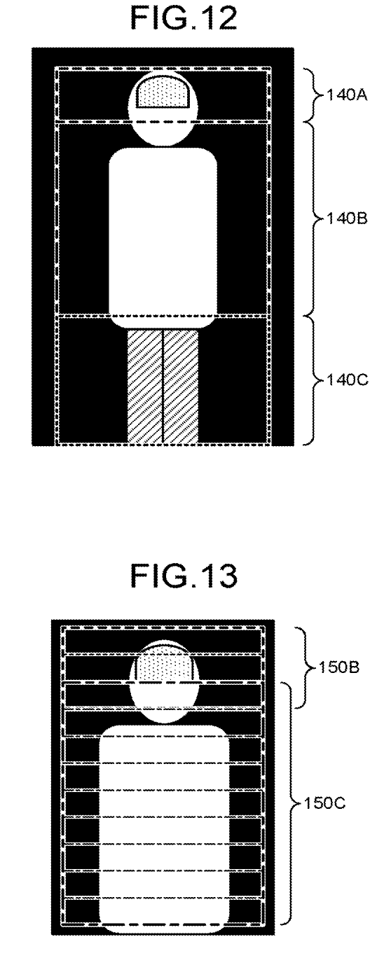

The processing executed by the medical image diagnosis system according to the sixth embodiment will be explained with reference to FIGS. 3 and 12. FIG. 12 is a diagram illustrating setting of a scan condition of the medical image diagnosis system according to the sixth embodiment.

In the sixth embodiment, similarly to the first embodiment, the processing illustrated in the flowchart in FIG. 3 is executed. Hereinafter, detailed explanation will be omitted with regard to execution of the processing similar to the first embodiment.

The second gantry 2 executes the scan using the continuous scan method. The processing circuit 5 accepts a predetermined parameter according to the continuous scan method as the scan condition. In Step S101, for example, the processing circuit 5 accepts, by the receiving function 35, an input of the total scan time duration “4 minutes” for a body part “head part” as the scan condition that is dependent on the body part of the subject. The processing circuit 5 accepts, by the receiving function 35, an input of the total scan time duration “1 minute” for a body part “lower extremity” as the scan condition that is dependent on the body part of the subject. The processing circuit 5 accepts, by the receiving function 35, an input of the total scan time duration “2 minutes” for a body part “others” as the scan condition that is dependent on the body part of the subject.

As illustrated in FIG. 12, in the sixth embodiment, by executing the processing similar to that of the first embodiment, the processing circuit 5 specifies, by the specifying function 33, a predetermined body part of the subject on the basis of the specified position of the feature point in Step S104. In the example illustrated in FIG. 12, the processing circuit 5 specifies, by the specifying function 33, a body part “head part” as the scanning range 140A. The processing circuit 5 specifies, by the specifying function 33, a body part “lower extremity” as the scanning range 140C. The processing circuit 5 specifies, by the specifying function 33, a body part “others” as the scanning range 140B.

The processing circuit 5 according to the sixth embodiment sets, by the setting function 32, a predetermined parameter according to the continuous scan method in Step S105.

The processing circuit 5 sets, by the setting function 32, the total scan time duration “4 minutes” as the scan condition in the scanning range 140A specified as the body part “head part”. Similarly, the processing circuit 5 sets, by the setting function 32, the total scan time duration “1 minute” as the scan condition in the scanning range 140C specified as the body part “lower extremity”. Similarly, the processing circuit 5 sets, by the setting function 32, the total scan time duration “2 minutes” as the scan condition in the scanning range 140B specified as the body part “others”. The processing circuit 5 executes the scan using the continuous scan method on the basis of the scan condition that is set in this way.

In this way, the processing circuit 5 according to the sixth embodiment sets, by the setting function 32, a predetermined parameter according to the continuous scan method. The image of the intended precision can be obtained for each of the body parts, in such a state that the precision of the generated image is spatially equalized, by the continuous scan method. However, if the operator manually sets a parameter of each body part in the continuous scan method, the burden of an operator is heavy. By employing the medical image diagnosis system according to the sixth embodiment, the burden of the operator can be lessened while maintaining benefit of the continuous scan method.

### Seventh Embodiment

In the seventh embodiment, the processing circuit 5 accepts, by the receiving function 35, an input of the reconstruction condition of the acquired data as the scan condition that is dependent on the body part (the scan condition of each body part). The processing circuit 5 sets, by the setting function 32, the scan condition of the second scan that is dependent on the body part, on the basis of the accepted input of the scan condition.

The processing executed by the medical image diagnosis system according to the seventh embodiment will be explained with reference to FIGS. 3 and 13. FIG. 13 is a diagram illustrating setting of a scan condition of the medical image diagnosis system according to the seventh embodiment.

In the seventh embodiment, similarly to the first embodiment, the processing illustrated in the flowchart in FIG. 3 is executed. Hereinafter, detailed explanation will be omitted with regard to execution of the processing similar to the first embodiment.

In Step S101, for example, the processing circuit 5 accepts, by the receiving function 35, for a body part “head part”, an input of a reconstruction condition of the head part as the scan condition that is dependent on the body part of the subject. The processing circuit 5 accepts, by the receiving function 35, for a body part “whole body (body parts except for head part)”, an input of a reconstruction condition of the whole body (body parts except for head part) as the scan condition that is dependent on the body part of the subject. Specific example of the reconstruction condition includes the number of iterations and the number of subsets of the aforementioned OSEM method.

As illustrated in FIG. 13, in the seventh embodiment, by executing the processing similar to that of the first embodiment, the processing circuit 5 specifies, by the specifying function 33, a predetermined body part of the subject on the basis of the specified position of the feature point in Step S104. In the example illustrated in FIG. 13, the processing circuit 5 specifies, by the specifying function 33, a body part “head part” as the scanning range 150B. The processing circuit 5 specifies, by the specifying function 33, a body part “whole body (body parts except for head part)” as the scanning range 150C.

The processing circuit 5 according to the seventh embodiment sets, by the setting function 32, a reconstruction condition of the data that is dependent on the body part as the scan condition in Step S105. Specifically, the processing circuit 5 sets, by the setting function 32, a reconstruction condition of the head part as the scan condition in the scanning range 150A specified as the body part “head part”. Similarly, the processing circuit 5 sets, by the setting function 32, an input of a reconstruction condition of the scan of the whole body (body parts except for head part) as the scan condition in the scanning range 150C specified as the body part “whole body (body parts except for head part)”.

The processing circuit 5 generates, in Step S109, a functional image on the basis of the reconstruction condition that is set in Step S105.

In this way, the processing circuit 5 according to the seventh embodiment sets, by the setting function 32, a reconstruction condition. Because required precision of the image is different for each body part, it is desirable that a reconstruction condition of the image be set for each of the body parts. However, if the operation of setting the parameter is manually executed by the operator, the burden of the operator is heavy. By employing the medical image diagnosis system according to the seventh embodiment, the burden of the operator can be lessened.

### Other Embodiments

In the medical image diagnosis system according to the embodiments, the case is explained in which the first gantry 1 includes the base of the X-ray CT scanning apparatus and the second gantry 2 includes the base of the PET apparatus. However, the modality is not limited to the embodiments described above. For example, the first gantry 1 may be a gantry that is included in a magnetic resonance imaging apparatus. The second gantry 2 may be a gantry that is included in a single photon emission computed tomography apparatus (SPECT apparatus).

In the medical image diagnosis system according to the embodiments, the structural image diagnosis apparatus may be independently extracted to configure the system. For example, the structural image diagnosis apparatus may extract a feature point from a structural image acquired by the scan to specify a body part, and, on the basis of the result, may set the scan condition that is dependent on the body part in the second scan to be executed by the functional image diagnosis apparatus. Specifically, the processing circuit 5 of the structural image diagnosis apparatus includes the first generating function 11, the specifying function 33, and the setting function 32. The processing circuit 5 generates, by the first generating function 11, a structural image on the basis of the first scan for the subject P. The processing circuit 5 specifies, by the specifying function 33, the position of the feature point of the subject P from the generated structural image, and further specifies a body part of the subject P on the basis of the specified position of the feature point. The processing circuit 5 sets, by the setting function 32, the scan condition of the second scan that is executed for generating a functional image that is dependent on the body part, for the body part specified by the specifying function 33.

The structural image diagnosis apparatus includes a gantry (for example, first gantry 1) and the processing circuit 5. The gantry collects a structural image. The processing circuit 5 acquires information on the position of a body part of the subject on the basis of the structural image that is acquired by the gantry. The processing circuit 5 controls the gantry, which acquires the nuclear medical image of the subject P, on the basis of the information on the position of a body part of the subject and the scan condition of each body part.

Moreover, in the medical image diagnosis system according to the embodiments, the functional image diagnosis apparatus (nuclear medical image diagnosis apparatus) may be independently extracted to configure the system. For example, the functional image diagnosis apparatus (nuclear medical image diagnosis apparatus) may extract a feature point from a structural image acquired from the structural image diagnosis apparatus to specify a body part of the subject, may set, on the basis of the result, the scan condition that is dependent on the body part, and may execute the scan to generate a functional image. The processing circuit 5 of the functional image diagnosis apparatus (nuclear medical image diagnosis apparatus) includes the specifying function 33, the setting function 32, and the second generating function 12. The processing circuit 5 specifies, by the specifying function 33, the position of the feature point of the subject P from a structural image generated by the first scan for the subject P, and further specifies a body part of the subject P on the basis of the specified position of the feature point. The processing circuit 5 sets, by the specifying function 33, a scan condition of the second scan that is dependent on the body part, for the specified body part. The processing circuit 5 generates, by the second generating function 12, a functional image on the basis of the second scan that is executed in accordance with a scan condition that is dependent on the body part.

The nuclear medical image diagnosis apparatus includes the processing circuit 5, a gantry (for example, second gantry 2). The processing circuit 5 acquires a structural image from the structural image diagnosis apparatus that collects a structural image inside the subject P. The gantry acquires the nuclear medical image of the subject. The processing circuit 5 acquires information on the position of a body part of the subject P on the basis of the structural image, and controls the gantry on the basis of information on the position of a body part and a scan condition of each body part.

According to at least one of the embodiments described above, the burden of an operator can be lessened.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnosis system that includes a structural image diagnosis apparatus and a nuclear medical image diagnosis apparatus, the system comprising:
a processing circuit configured to acquire information on a position of a body part of a subject based on a structural image acquired by the structural image diagnosis apparatus; and
a gantry configured to scan a nuclear medical image of the subject, wherein
the processing circuit is configured to control the gantry based on the information on the position of the body part and a scan condition of the body part.

2. The medical image diagnosis system according to claim 1, wherein the processing circuit is
configured to specify a position of a feature point of the subject from the structural image,
configured to specify a body part of the subject based on the specified position of the feature point,
configured to set a scan condition of a scan by the gantry,
configured to control the gantry based on information on a position of the specified body part and a scan condition that is set for the specified body part, and
configured to generate a nuclear medical image based on the scan executed in accordance with the scan condition.

3. The medical image diagnosis system according to claim 1, further comprising a memory circuit configured to memorize the scan condition of the body part in a scan by the gantry.

4. The medical image diagnosis system according to claim 1, wherein the processing circuit is
configured to accept an input of the scan condition that is dependent on the body part, and
configured to set the scan condition based on the accepted input of the scan condition.

5. The medical image diagnosis system according to claim 2, wherein the processing circuit is
configured to specify positions of feature points of the subject from the structural image,
configured to specify a plurality of body parts of the subject based on the specified positions of the feature points,
configured to set, for each of the plurality of specified body parts, a scan condition of the scan that is dependent on each of the plurality of body parts, and
configured to generate the nuclear medical image based on the scan that is executed in accordance with the scan condition that is dependent on each of the plurality of body parts.

6. The medical image diagnosis system according to claim 1, wherein the processing circuit accepts an input of a scan time duration or a scan speed as the scan condition of the body part.

7. The medical image diagnosis system according to claim 4, wherein, in a case in which, for a body part, the scan is divided into a plurality of times of scans and the plurality of times of scans is executed, the processing circuit is configured to accept an input of a size of an overlapped area in each scan as the scan condition of the body part.

8. The medical image diagnosis system according to claim 4, wherein the processing circuit is configured to accept an input of a reconstruction condition of acquired data as the scan condition of the body part.

9. The medical image diagnosis system according to claim 4, wherein the processing circuit is configured to accept an input of a scan condition of a synchronous scan as the scan condition of the body part.

10. The medical image diagnosis system according to claim 4, wherein the processing circuit is configured to accept an input of a scan condition of a dynamic scan as the scan condition of the body part.

11. The medical image diagnosis system according to claim 1, wherein the processing circuit is configured to set a size of Field Of View (FOV) as the scan condition of the body part.

12. The medical image diagnosis system according to claim 1, wherein the scan includes a continuous scan that is performed while continuously moving a body part of the subject to be scanned.

13. A nuclear medical image diagnosis apparatus comprising:
a processing circuit configured to acquire a structural image of a subject from a structural image diagnosis apparatus acquiring the structural image; and
a gantry configured to scan a nuclear medical image of the subject, wherein
the processing circuit is
configured to acquire information on a position of a body part of the subject based on the structural image and
configured to control the gantry based on the information on the position of the body part and a scan condition of the body part.

14. A structural image diagnosis apparatus comprising;
a gantry configured to collect a structural image; and
a processing circuit configured to acquire information on a position of a body part of a subject based on the structural image acquired by the gantry, wherein
the processing circuit is configured to control the gantry scanning a nuclear medical image of the subject, based on the information on the position of the body part and a scan condition of the body part.
